(19) United States
(12) Patent Application Publication
(10) Pub. No.: US 2023/0018521 A1
(43) Pub. Date: Jan. 19, 2023

(54) SYSTEMS AND METHODS FOR GENERATING TARGETED OUTPUTS

(71) Applicant: P3 Health Partners, Henderson, NV (US)

(72) Inventors: Rebecca Lindy, Tucson, AZ (US); James A. Henderson, JR., Henderson, NV (US); Unmesh SRIVASTAVA, Monrovia, CA (US)

(73) Assignee: P3 Health Partners, Henderson, NV (US)

(21) Appl. No.: 17/657,376

(22) Filed: Mar. 31, 2022

Related U.S. Application Data
(60) Provisional application No. 63/169,371, filed on Apr. 1, 2021.

(51) Int. Cl. G16H 50/20; G16H 50/30; G16H 40/20

(52) U.S. Cl. CPC G16H 50/20; G16H 50/30; G16H 40/20

(57) ABSTRACT

A method for implementing a targeted medical outputs is disclosed. The method may comprise: receiving first user data associated with a user from an external server; receiving second user data associated with the user from an internal server; processing the first user data and the second user data at a data fabric structure to generate domains; receiving the domains as an input at a machine learning model trained to generate a machine learning output; receiving at a database cube the machine learning output; performing one or more database cube processing operations on the machine learning output to generate a database cube output; transmitting the database cube output to one or more outputs, identified based on the one or more markers.

### CROSS-REFERENCE TO RELATED APPLICATIONS

This application claims priority to U.S. Provisional Application No. 63/169,371 filed Apr. 1, 2021, the entire disclosure of which is hereby incorporated herein by reference in its entirety.

### TECHNICAL FIELD

Various embodiments of the present disclosure relate generally to targeted medical outputs, and more particularly to, systems and methods for implementing an output system whereby users are automatically informed about health risks or probabilities, based on user-specific information.

### BACKGROUND

Health care is generally conducted by a local medical provider. Generally, a medical provider only has access to patient data that the medical provider has collected. However, patients often have more than one medical provider. Different medical providers may collect different information from the same patient and each medical provider, without knowing all of the patient's information, may come to inaccurate decisions as to diagnosis, treatment, etc. Further, medical providers may have to repeat tests due to this lack of communication, creating duplicate expenses for the patient and the health care system. This lack of a cohesive system generally results in providers being unaware of patients' statuses when they receive medical care outside of their office. As such, holistic care and treatment for patients may be ineffective.

Conventional techniques, including the foregoing, generally fail to monitor patient care outside of a given medical provider's office, stifling medical outputs. When one medical provider is unaware of the patient's status or another medical provider's data, medical care cannot be effectively implemented. This delay of information can generally make treatment less effective and pose health risks to patients due to delays in diagnosis, treatment, etc.

The background description provided herein is for the purpose of generally presenting the context of the disclosure. Unless otherwise indicated herein, the materials described in this section are not prior art to the claims in this application and are not admitted to be prior art, or suggestions of the prior art, by inclusion in this section.

### SUMMARY OF THE DISCLOSURE

According to certain aspects of the disclosure, methods and systems are disclosed for targeted medical outputs.

In one aspect, an exemplary embodiment of a method for targeted medical outputs may include: receiving first user data associated with a user from an external server via a first secure network connection; receiving second user data associated with a user from an internal server via a second secure network connection; processing the first user data and the second user data at a data fabric structure to generate domains; receiving the domains as an input at a machine learning model trained to correlate the domains with one or more users and to generate a machine learning output, for each of the one or more users, the machine learning output comprising correlated data for each of the one or more users and one or more markers associated with the correlated data for each of the one or more users; receiving, at the database cube and for each of the one or more users, the machine learning output; performing one or more database cube processing operations on the machine learning output to generate a database cube output, the database cube output based at least on the one or more markers associated with the correlated data; transmitting the database cube output to one or more outputs, wherein the one or more outputs are identified based on the one or more markers.

In another aspect, an exemplary embodiment of a system for implementing targeted medical outreach may include: transmitting the database cube output to a user device, the user device configured to respond to the database cube output to generate response data using one or more sensors; receiving the response data output from the user device; providing the response data to the machine learning model, the machine learning model configured to generate an updated machine learning output based on the response data, wherein the updated machine learning output comprises an updated marker.

In another aspect, an exemplary embodiment of a system for implementing targeted medical outreach may include: receive second user data associated with a user from an internal server via a second secure network connection; process the first user data and the second user data at a data fabric structure to generate domains; receive the domains as an input at a machine learning model trained to correlate the domains with one or more users and to generate a machine learning output, for each of the one or more users, the machine learning output comprising correlated data for each of the one or more users and one or more markers associated with the correlated data for each of the one or more users; receive, at a database cube and for each of the one or more users, the machine learning output; perform one or more database cube processing operations on the machine learning output to generate a database cube output, the database cube output based at least on the one or more markers associated with the correlated data; transmit the database cube output to one or more outputs, wherein the one or more outputs are identified based on the one or more markers.

In another aspect, an exemplary embodiment of a method for targeted medical outreach may include: receiving first user data associated with a user, from a plurality of users, from an external server via a first secure network connection; receiving second user data associated with the user from an internal server via a second secure network connection; processing the first user data and the second user data at a data fabric structure to generate domains; receiving the domains as an input at a database cube for each of the plurality of users including the user; performing one or more database cube processing operations on the domains to generate a database cube output, the database cube output based at least on the one or more markers associated with the correlated data; transmitting the database cube output to one or more outputs, wherein the one or more outputs are identified based on the one or more markers.

It is to be understood that both the foregoing general description and the following detailed description are exemplary and explanatory only and are not restrictive of the disclosed embodiments, as claimed.

### BRIEF DESCRIPTION OF THE DRAWINGS

The accompanying drawings, which are incorporated in and constitute a part of this specification, illustrate various exemplary embodiments and together with the description, serve to explain the principles of the disclosed embodiments.

FIG. 1 depicts an exemplary environment for generating secure targeted medical outputs, according to one or more embodiments.

FIG. 2 depicts a flowchart of an exemplary method for generating secure targeted medical outputs, according to one or more embodiments.

FIG. 3A depicts an exemplary environment for generating secure targeted medical outputs for a patient with a patient wearable, according to one or more embodiments.

FIG. 3B depicts an exemplary environment for generating secure targeted medical outputs for a physician, according to one or more embodiments.

FIG. 4 illustrates an exemplary targeted medical output received by a patient, according to one or more embodiments.

FIG. 5 depicts an example of training a machine learning model, according to one or more embodiments.

FIG. 6 depicts an example of a computing device, according to one or more embodiments.

### DETAILED DESCRIPTION OF EMBODIMENTS

Generally, medical providers find it difficult to monitor patient health and status information outside of a given medical provider's office. Accordingly, improvements in technology relating to implementing targeted medical outputs are needed.

According to certain aspects of the disclosure, methods and systems are disclosed for generating secure targeted medical outputs. These methods and systems may improve holistic tracking, care, and management of patients across various medical providers. As discussed in more detail below, in various embodiments, systems and methods are described for using machine learning to implement targeted medical outputs. By training a machine learning model, e.g., via supervised, semi-supervised, or unsupervised learning, to learn associations between correlated data associated with one or more users and one or more markers, the trained machine learning model may be used to track patient health and predict health outcomes in response to input of user data and/or modified user data.

Reference to any particular activity is provided in this disclosure only for convenience and not intended to limit the disclosure. A person of ordinary skill in the art would recognize that the concepts underlying the disclosed devices and methods may be utilized in any suitable activity. The disclosure may be understood with reference to the following description and the appended drawings, wherein like elements are referred to with the same reference numerals.

The terminology used below may be interpreted in its broadest reasonable manner, even though it is being used in conjunction with a detailed description of certain specific examples of the present disclosure. Indeed, certain terms may even be emphasized below; however, any terminology intended to be interpreted in any restricted manner will be overtly and specifically defined as such in this Detailed Description section. Both the foregoing general description and the following detailed description are exemplary and explanatory only and are not restrictive of the features, as claimed.

In this disclosure, the term “based on” means “based at least in part on.” The singular forms “a,” “an,” and “the” include plural referents unless the context dictates otherwise. The term “exemplary” is used in the sense of “example” rather than “ideal.” The terms “comprises,” “comprising,” “includes,” “including,” or other variations thereof, are intended to cover a non-exclusive inclusion such that a process, method, or product that comprises a list of elements does not necessarily include only those elements, but may include other elements not expressly listed or inherent to such a process, method, article, or apparatus. The term “or” is used disjunctively, such that “at least one of A or B” includes, (A), (B), (A and A), (A and B), etc. Relative terms, such as, “substantially”, “approximately”, or “generally,” are used to indicate a possible variation of ±10% of a stated or understood value.

It will also be understood that, although the terms first, second, third, etc. are, in some instances, used herein to describe various elements, these elements should not be limited by these terms. These terms are only used to distinguish one element from another. For example, a first user data could be termed a second user data, and, similarly, a second user data could be termed a first user data, without departing from the scope of the various described embodiments. The first contact and the second contact are both contacts, but they are not the same contact.

As used herein, the term if is, optionally, construed to mean “when” or “upon” or “in response to determining” or “in response to detecting,” depending on the context. Similarly, the phrase “if it is determined” or “if [a stated condition or event] is detected” is, optionally, construed to mean “upon determining” or “in response to determining” or “upon detecting [the stated condition or event]” or “in response to detecting [the stated condition or event],” depending on the context.

Terms like “provider,” “medical provider,” or the like generally encompass an entity, person, or organization that may seek information, resolution of an issue, or engage in any other type of interaction with a patient, e.g., to provide medical care, medical intervention or advice, or the like. Terms like “patient,” “client,” or the like generally encompass any person or entity who's data is applied, who is obtaining information, seeking resolution of an issue, or engaging in any other type of interaction with a medical provider, e.g., to receive such care, information, or the like. Terms like “user” generally encompass any person or entity that may obtain information, resolution of an issue, purchase of a product, or engage in any other type of interaction with a provider or patient.

As used herein, terms such as “marker” or the like generally encompass values that act as flags, signals, or identifiers for a function or process. The value or attribute of a marker may be used to determine the next step of a program.

As used herein, a “machine-learning model” generally encompasses instructions, data, and/or a model configured to receive input, and apply one or more of a weight, bias, classification, or analysis on the input to generate an output. The output may include, for example, a classification of the input, an analysis based on the input, a design, process, prediction, or recommendation associated with the input, or any other suitable type of output. A machine-learning model is generally trained using training data (e.g., historical user data, experiential data, and/or samples of input data), which are fed into the model in order to establish, tune, or modify one or more aspects of the model, e.g., the weights, biases, criteria for forming classifications or clusters, or the like. Aspects of a machine-learning model may operate on an input linearly, in parallel, via a network (e.g., a neural network), or via any suitable configuration.

The execution of the machine-learning model may include deployment of one or more machine learning techniques, such as linear regression, logistical regression, random forest, gradient boosted machine (“GBM”), deep learning, and/or a deep neural network. Supervised, semi-supervised, and/or unsupervised training may be employed. For example, supervised learning may include providing training data and labels corresponding to the training data, e.g., as ground truth. Unsupervised approaches may include clustering, classification or the like. K-means clustering or K-Nearest Neighbors may also be used, which may be supervised or unsupervised. Combinations of K-Nearest Neighbors and an unsupervised cluster technique may also be used. Any suitable type of training may be used, e.g., stochastic, gradient boosted, random seeded, recursive, epoch or batch-based, etc.

According to an example of the disclosed subject matter, a patient may elect or may be automatically enrolled to receive automated suggestions based on health risk factors (e.g., risk of stroke). Conventionally, a patient may visit separate medical providers, who may or may not communicate test results, diagnosis, physical findings, or other data with the other medical providers. Further, due to the subjective nature of human medical result interpretation, risk assessment by medical providers may generally be less reliable. Medical providers may not have a patient's full medical history, current medical status, and/or medical-related updates due to lack of communication between medical providers. Due to the lack of such information, risk assessment is generally subjective, and patients may not receive adequate information about current or future medical conditions. Thus, it is desirable to improve risk assessment, e.g., by implementing targeted medical outputs using various data processing technologies in combination with machine learning, as disclosed herein.

According to an example implementation, targeted medical outputs may be provided to a patient for automated risk assessment, e.g. using an outreach system that warns a patient of calculated health risks. A patient may receive targeted medical outreach about her health outcomes or other relevant data. One or more data servers (e.g., internal or external) may have access to data that can be transmitted to a data fabric. External patient data may be obtained from an external sever associated with one or more health institutions, insurance companies, and/or healthcare providers (e.g., Cigna, Aetna, Anthem, Blue Cross Blue Shield, and so forth). An internal server for the data fabric system may also receive, store, and/or provide other patient-related information, for example, information related to a patient's claims or payments. The data may be stored on a cloud-based data lake associated with a data fabric system, for example, Microsoft Azure Data Lake. The transmission of data from the external and/or internal servers may be from a trusted entity (e.g., a first party system) and/or an untrusted entity (e.g., a third party system). The one or more servers or one or more components (e.g., an application programming interface (“API”), a software development kit (“SDK”), etc.) may trigger a communication of data to the data fabric. Patient data from the cloud-based data lake and the internal database may then be imported to a staging table where it is prepared for integration. At this step, patient data may be modified, for example, by formatting the data into a better format for aggregation. As an example, data for a particular patient may be associated with multiple different member IDs; the patient data may be parsed and mapped to a preferred member ID for easier reference downstream. After the patient data is modified, relevant information may then be extracted from the modified data. For example, patient contact information might be considered relevant, and this data may be extracted. Similarly, information related to a recent hospital visit (visit date, discharge date and time, diagnoses, prescriptions, testing scheduled, and so forth) may each be found relevant and also extracted from the modified data. In some cases, a trained machine learning model may be trained to extract the data. In some embodiments, exception handling may be performed; for example, if it is clear that there is an error in the data (for example, there are 5000 new patients being added to the system during a routine update), the system may determine that a threshold is exceeded such that the data should be rejected. In that case, instead of loading new data, previously used or “old” data may temporarily be used until new data is received. The relevant data may then be extracted and parsed to generate atomic flattened data. This atomic data is then classified into one or more domains or subdomains. For example, the atomic data may be placed into the lab results domain, or the medical claim domain. One or more of the domains or atomic data may be transmitted to a machine learning model.

In some embodiments, the machine learning model may receive data from the data fabric. The machine learning model may use various techniques, discussed in more detail below, to analyze the received data. The machine learning model may output machine learning data which may be transmitted to a database cube (hereinafter referred to as “cube”) for processing. In any case, the data, results, or outputs may be transmitted via a secure network or any other suitable means.

As used herein, a database cube may be used to represent data along some dimensions of interest. For example, in an online analytical processing (“OLAP”) database cube, such dimensions could be the health data associated with a given user, the user's demographic information, and recent updates to the user's health data. In this setup, a fact would be a health event where a particular health care service has been provided in a particular health care subsidiary at a particular time. A data image time series dimensions would include latitude and longitude coordinates and time. A health fact or measure may be a pixel at a given space and time as recorded in the health data. A database cube may be a multi-dimensional concept which can be 1-dimensional, 2-dimensional, 3-dimensional, or higher-dimensional. According to implementations, every dimension of a database cube may divide data into groups of cells whereas each cell in the database cube represents a single measure of interest.

In some embodiments, the association between the data (e.g., data fabric data) and any information relevant to medical treatment (e.g., risk factor for disease development) may be predetermined, e.g., generated manually such as via one or more criteria. In some embodiments, such association may be at least partially learned via a machine learning process. For example, data fabric outputs (e.g., domains) and medical information (e.g., preexisting conditions, medical history, demographic information, etc.) may be used as training data to train a machine learning model, so that the trained model is configured to output one or more risk factors in response to input of one or more data fabric outputs. In an exemplary embodiment, a machine learning model may be initialized based on predetermined associations, and may thereafter be tuned or modified based on further data. In some embodiments, the training data may include data fabric outputs and/or historical user data.

In some embodiments, a sequence of data (e.g., data fabric outputs) and/or a sequence of analyses resulting from such data may be used to train a machine learning model and/or determine information regarding a patient. For example, successive data fabric outputs over time may be used, e.g., in an intra-patient manner, to compare a patient's outputs to a baseline for the patient, or to track, identify, or predict changes over time.

In another example, in an inter-patient manner, successive data fabric outputs from multiple patients, alongside medical information regarding the onset or change in one or more medical results or conditions, may be used to identify associations between the data fabric outputs and the medical results or conditions that can be used to predict various diagnoses, concerns, etc. in a patient. In other words, a change over time evident in multiple patient data fabric outputs may coincide with those same patients having a medical or physiological change, and thus the aspect evident in the data fabric outputs may then be used to predict or diagnose that medical or physiological change in future patients.

In some embodiments, the data from the machine learning model may be received at a database cube. The database cube may use various techniques, discussed in more detail below, to analyze the received data, e.g., by generating markers based on inputted data. The database cube may output data which may be transmitted to one or more outputs (e.g., recipients). Before transmitting the data to the output, the database cube may mark certain outputs based on which recipient outputs should receive the information, e.g., a physician and not a patient. The one or more outputs may be, for example, an internal report generation module (e.g., an internal reporting system implemented using a processor and report generation algorithm), an automated outreach to a patient or physician, a health care provider graphical user interface, etc. The data, results, and/or outputs may be transmitted via a secure network or any other suitable means.

A secure network may be implemented using policies, processes, and practices adopted to prevent, detect, and monitor unauthorized access, misuse, modification, or denial of a computer network and network-accessible resources. A secure network may be implemented using encryption technology and/or access credentials communicated between, for example, database cubes and/or output modules.

While several of the examples above involve targeted medical outputs to predict disease development, it should be understood that techniques according to this disclosure may be adapted to recommend medical treatments, explain risk assessment, predict disease relapse, etc. It should also be understood that the examples above are illustrative only. The techniques and technologies of this disclosure may be adapted to any suitable activity.

Disclosed below are various systems and methods of implementing targeted data outputs using data manipulation and machine learning techniques. As will be discussed in more detail below, these systems and methods may include one or more aspects according to this disclosure that may be apparent to one of ordinary skill in the art.

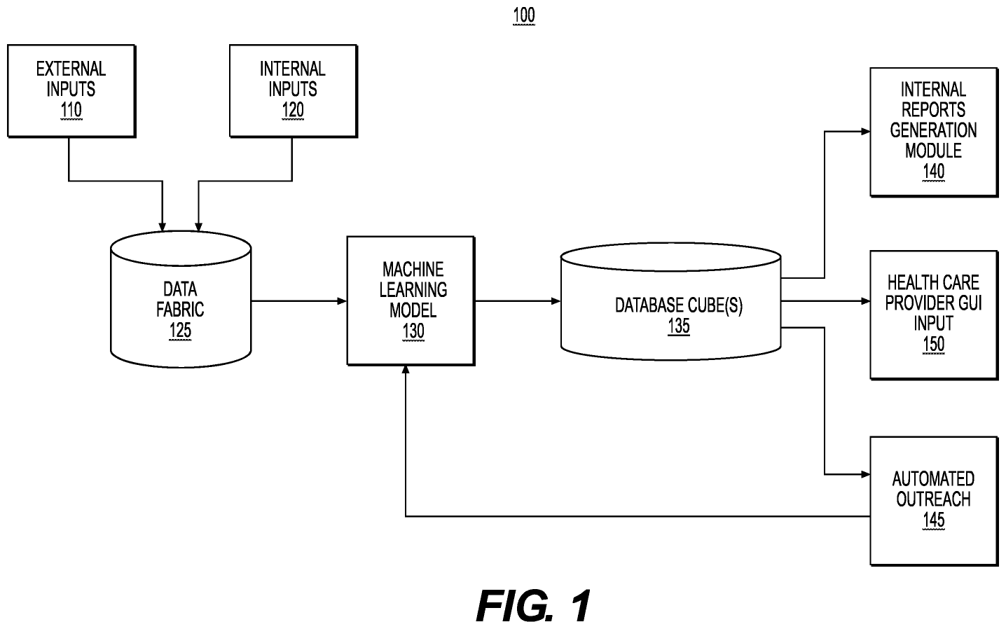

FIG. 1 depicts an exemplary environment 100 that may be utilized with techniques presented herein. One or more of external inputs 110 and/or internal inputs 120, a data fabric 125, a machine learning model 130, a database cube 135, and an output may communicate by any suitable means. As will be discussed below, the output from database cube 135 may be one or more of an internal report generation module 140 (e.g., implemented using a processor and report generation algorithm), an automated outreach 145 module (e.g., implemented using a processor and an outreach algorithm), a health care provider graphical user interface 150, and/or any other suitable output.

Data fabric 125 may receive user data (e.g., external inputs 110 and internal inputs 120) via a secure file transfer process from external databases, such as a secure hypertext transfer protocol (“S-HTTP”). According to some aspects, the user data comprises one or more of: user institution records, user identification information, or user financial data. User data, as described in further detail below, may include any data relevant to a patient or user's health, medical records, or medical history. Examples include diagnoses, lab test results, x-ray results, emergency room visits and discharge records, hospital or clinic admission information, and other information relevant to the health, medical treatment, and well-being of a user or patient. According to some aspects, the user data is in the format of one or more of: an .xls file; a csv file; a pipe delimited file, or a text file. The user data may be stored on a data lake, for example a cloud-based data lake such a Microsoft Azure Data Lake.

External inputs 110 may include various sources of data, such as an external server. External inputs 110 may include various sources of data, such as an external sever associated with one or more health institutions, insurance companies, and/or healthcare providers (e.g., Cigna, Aetna, Anthem, Blue Cross Blue Shield, and so forth). Internal inputs 120 may include various sources of data, such as an internal server. The internal server may hold patient-related information, such as internal information related to a patient's claims or payments or any other relevant information. In any case, external inputs 110 and/or internal inputs 120 may be transmitted to other aspects of the environment, such as to data fabric 125, from a trusted entity (e.g., a first party system) and/or an untrusted entity (e.g., a third party system). The one or more external and/or internal servers or one or more components (e.g., an application programming interface (“API”), a software development kit (“SDK”), etc.) may trigger a communication of external inputs 110 and internal inputs 120 to data fabric 125.

External databases may be associated with one or more health institutions or insurers (e.g., Allwell, Humana, Cigna, Healthnet, and so forth). Additional data from an internal server may also be stored on the data lake, such as internal data relevant to a patient, for example, patient payment information or credit score information. Data stored on the data lake may be stored in its original format, e.g., .xls, .csv, text, pipe delimited, and other typical formats. Once user data (e.g. patient data) is collected on the data lake, it may then be sent to a staging process, where the data is temporarily placed into one or more staging tables. Once the user data is received at the staging tables, it is processed for integration. While the data at the staging table in some embodiments is kept in its original format, in other embodiments, the data may be formatted into another file format. Once the data is placed on the table, it may be processed or modified. For example, data on the table may be mapped to other data received from an internal database or other source. For example, the source data may have multiple different member IDs for the same patient, but the data fabric relies on only one type of member ID. The user data may thus be modified and mapped to the optimal member ID for further processing. Relevant data may then be extracted from the modified user data. Further, during this process, the data may be audited. For example, if the data indicates that 5,000 new patients are being added for a particular health care provider, the significant change in size may indicate an error in the received data. When an error is detected, the new data may be rejected. In some cases, upon auditing detecting an error, previously used relevant data or a version thereof may be used. In this manner, a previously saved version of the relevant data will remain accessible to a healthcare provider and data analytics downstream even if the newly received data from the data lake contains errors. Once relevant user data has been extracted the relevant data may be further parsed at integration to generate atomic flattened data, e.g., data that has been broken down into its smallest logical unit. For example, data such as a price, a zip code, or a street address number may be atomic data. On the other hand, data such as an entire street address (which has multiple fields) would not be considered atomic data. Once the atomic data has been obtained, it may then be sorted into one or more domains. Domains may include, for example, a provider domain, lab results domain, medical claim domain, member domain, and so forth. By sorting the atomic data into a plurality of domains, the data output from data fabric 125 (“data fabric output”) can now be easily and quickly retrieved by one or more analytics processes, for example, targeted medical outreach. In this manner, an improved data fabric 125 is provided that results in a demonstrably improved software for healthcare providers and patients.

In some embodiments, machine learning model 130 may analyze data from another component of environment 100, such as data fabric output. Machine learning model 130 may also, as discussed in more detail below, analyze data received from a patient wearable. In some embodiments, machine learning model 130 may receive additional data, e.g., medical, demographic, or personal information for a patient, etc. For example, in some embodiments, a medical provider may specify a particular condition or characteristic of interest for a patient to machine learning model 130.

Machine learning model 130 may process the data it receives using various machine learning techniques, discussed in more detail below. In some embodiments, the data output by machine learning model 130 (“machine learning data”) may be transmitted to one or more database cubes 135, another component of environment 100, or any other suitable person, entity, or device. Machine learning data may be risk values, disease probabilities, disease types, trends in disease probabilities, relapse probabilities, or any other value that may be used to track and/or manage patient health.

In some embodiments, the machine learning data may be the data fabric output correlated with a given user (e.g., medical, diagnostic, or other health-related information or results) and associated with markers. The machine learning data may comprise predictions for a patient's hospital or emergency room admission or readmission. The markers of machine learning model 130 (“machine learning markers”) may be based thresholds for various blood tests, visualizations on scans, or any other marker that may be relevant to patient outreach. A marker may be an indication generated based on, for example, exceeding a threshold. A marker may indicate if a given health concern, a risk factor, a trend, or the like is above a threshold amount. The threshold amount may be determined by machine learning model 130. The threshold amounts for different markers (e.g., disease designations, risk value, trend, etc.) may be initialized based on training a machine learning model and may be updated based on ongoing data inputs associated with the model.

For example, machine learning model 130 may receive conflicting or inconclusive data fabric outputs relating to blood markers for cholecystitis and a Hepatobiliary Iminodiacetic Acid (“HIDA”) Scan. A HIDA scan is conducted by injecting a small amount of radioactive tracer into the patient's blood stream so that the gallbladder can be visualized using a gamma camera. In this example, blood test results may show that the patient has high levels of certain blood markers (e.g. white blood cells, alkaline phosphatase, and bilirubin elevated above a threshold amount), while the HIDA scan may show that the radioactive tracer hardly entered the gallbladder, and what did enter the gallbladder, left the organ in amounts much lower than expected. The blood test results may be highly indicative of an issue with the gallbladder, but the HIDA scan may be inconclusive as to whether the issue is acute or chronic cholecystitis. According to this example, machine learning model 130 may generate one or more machine learning markers, based on blood values above a certain threshold in combination with HIDA scan results, to mark these results. Based on applying the data to applicable weights, biases, and/or layers, machine learning model 130 may generate a possible diagnosis (e.g., a percent likelihood the patient may have acute or chronic cholecystitis), recommendations (e.g., seek immediate medical care), a suggested treatment plan, either short-term (e.g., consume a low-fat diet) or long-term (e.g., removal of the gallbladder), and/or any other relevant information using the output machine learning markers. For example, machine learning model 130 may output results including headers comprising one or more markers (e.g., bit values) that indicate one or more of the possible diagnoses (e.g., a percent likelihood the patient may have acute or chronic cholecystitis), recommendations (e.g., seek immediate medical care), a suggested treatment plan, either short-term (e.g., consumer a low-fat diet) or long-term (e.g., removal of the gallbladder), and/or any other relevant information.

In some embodiments, various components of environment 100 may generate, store, train, or use machine learning model 130 and/or may include instructions associated with machine learning model 130, e.g., instructions for generating machine learning model 130, training machine learning model 130, using machine learning model 130, etc. Various components of environment 100 may include instructions for retrieving user data (e.g., external inputs 110 or internal inputs 120), including historical user data, and/or adjusting the user data, e.g., based on the data fabric output. Historical user data may include data generated either manually or automatically within an enterprise. Various components of environment 100 may include training data, e.g., data fabric training data and/or additional training data obtained by applying one or more algorithms or processes relating to data fabric 125 to training external inputs or training internal inputs from one or more individuals, and may include ground truth, e.g., diagnostic or pathophysiologic training data associated with the one or more individuals.

In some embodiments, a system or device other than those in environment 100 may be used to generate and/or train machine learning model 130. For example, such a system may include instructions for generating machine learning model 130, the training data and ground truth, and/or instructions for training machine learning model 130. A resulting trained machine learning model 130 may then be provided to environment 100.

Generally, a machine learning model includes a set of variables, e.g., nodes, neurons, filters, etc., that are tuned, e.g., weighted or biased, to different values via the application of training data. In supervised learning, e.g., where a ground truth is known for the training data provided, training may proceed by feeding a sample of training data into a model with variables set at initialized values, e.g., at random, based on Gaussian noise, a pre-trained model, or the like. The output may be compared with the ground truth to determine an error, which may then be back-propagated through the model to adjust the values of the variable.

Training may be conducted in any suitable manner, e.g., in batches, and may include any suitable training methodology, e.g., stochastic or non-stochastic gradient descent, gradient boosting, random forest, etc. In some embodiments, a portion of the training data may be withheld during training and/or used to validate the trained machine-learning model, e.g., compare the output of the trained model with the ground truth for that portion of the training data. The training of machine learning model 130 may be configured to cause machine learning model 130 to learn associations between (i) external inputs 110, internal inputs 120, and/or data fabric outputs and (ii) diagnostic or pathophysiologic data, such that the trained machine learning model is configured to determine an output (e.g., diagnosis, notification, or alert) in response to the input data based on the learned associations. For example, machine learning model 130 may receive an input of data fabric data indicating a strong correlation between facial numbness and subsequent hospitalizations which machine learning model 130 may learn to recognize as predictive of a stroke.

In some embodiments, the variables of machine learning model 130 may be interrelated in any suitable arrangement in order to generate an output. Output data may be tagged using a given result associated with the data, a header, metadata, or using any other suitable tag. For example, in some embodiments, machine learning model 130 may include architecture that is configured to identify, isolate, and/or extract features, geometry, and or structure in input data. In another example, machine learning model 130 may include one or more convolutional neural networks (“CNN”) configured to identify features in the signal-processed data, and may include further architecture, e.g., a connected layer, neural network, etc., configured to determine a relationship between the identified features in order to determine a location in the signal-processed data.

In some instances, different samples of training data and/or input data may not be independent. In some embodiments, machine learning model 130 may include previous data fabric outputs to generate a result. For example, machine learning model 130 may be configured to consider various portions of the input data (e.g., data fabric outputs) in conjunction with each other. Thus, in some embodiments, machine learning model 130 may be configured to account for and/or determine relationships between multiple samples. In other words, an earlier portion of data fabric outputs may impact the ways a later portion is evaluated, or vice versa.

For example, in some embodiments, machine learning model 130 may include a Recurrent Neural Network (“RNN”). Generally, RNNs are a class of feed-forward neural networks that may be well adapted to processing a sequence of inputs. In some embodiments, machine learning model 130 may include a Long Shor Term Memory (“LSTM”) model and/or Sequence to Sequence (“Seq2Seq”) model. An LSTM model may be configured to generate an output from a sample that takes at least some previous samples and/or outputs into account. For example, a Seq2Seq model may be configured to receive a sequence of data fabric results of a particular time frame to identify the number of hospitalizations over time, indicative of a medical condition coinciding with a period of time in which the sequence was captured, and the identified change may thereafter be applied to new data that may include a single sample of data fabric outputs or a sequence of data fabric outputs.

In some embodiments, one or more database cubes 135 may receive and/or process machine learning data or other data from any component or output of environment 100. One or more database cubes 135 may use any suitable algorithm, method, or process for analyzing the data received by one or more database cubes 135, e.g. an OLAP structure. One or more database cubes 135 may use any suitable language to analyze data, such as Formula Translation (“Fortran”), Array Processing Language (“APL”), Interactive Data Language (“IDL”), Numerical Python (“NumPy”), Program Design Logic (“PDL”), and/or S-Lang. In some embodiments, database cube 135 may also be an algorithm that processes input data, e.g. machine learning data, to generate an output. The output from the algorithm may be similar or different to the output from the database cube 135, discussed below.

Database cube 135 may generate an output (“cube output”) that may be based on relevant fact data, such as machine learning outputs, associated with one or more markers. Database cube outputs may include machine-readable files that store relevant fact data, such as the user data correlated with markers. For example, the database cube output may comprise the patient's medical data correlated with their location and/or urgency values. The database cube markers may measure the urgency of the machine learning outputs and determine which parties to send the database cube output based on that urgency. For example, high urgency database cube outputs may be sent to both the medical provider and the patient. In another example, low urgency data may be sent to only the patient. Database cube 135 may generate one or more urgency values and may determine one or more entities to provide an output to, based on whether an urgency value meets a threshold amount or falls within a range.

Database cube 135 may determine the level of urgency based on the received machine learning outputs. For example, database cube 135 may receive a machine learning output that includes markers indicating a high probability of an acute cholecystitis diagnosis with a high risk assessment. Database cube 135 may use the marker to mark this output as highly urgent (e.g., an urgency value above a threshold), which may trigger database cube 135 to send the database cube output to both the medical provider and the patient.

In another example, database cube 135 may receive a machine learning output that includes a marker indicating a low probability of a diagnosis of acute cholecystitis, but a high likelihood of a diagnosis of acid reflux with a low risk assessment. Database cube 135 may use the marker to mark this input of low urgency, which may trigger database cube 135 to send the data to only the patient with the recommendation from the machine learning output for the patient to make an appointment with a medical provider. In any case, database cube 135 may be configured to send information to one or more parties. The parties may be able to program database cube 135 to send outputs based on the parties' preferences.

One or more database cubes 135 may communicate with other components of environment 100 by any suitable means, as discussed below. The database cube output may be one or more outputs, such as internal report generation module 140, automated outreach 145, health care provider graphical user interface 150, and/or any other suitable output.

Internal report generation module 140 may receive the database cube output. Internal report generation module 140 may generate a report based on the database cube output. The report may sort, store, or otherwise use the data to update the one or more internal servers. The report may be housed internally or externally.

Automated outreach 145, discussed in more detail below, may send information to patients, for example, suggested treatment plans. In some embodiments, discussed in greater detail below, automated outreach 145 may communicate with machine learning model 130. Automated outreach 145 may transmit data, such as health outcomes of a patient, to machine learning model 130. Machine learning model 130 may then alter its risk assessment for the particular patient.

Health care provider graphical user interface 150 may receive the database cube output. A graphical user interface (“GUI”) is a form of user interface that allows users to interact with electronic devices through graphical icons and/or audio indicator instead of text-based user interface, typed command labels, or text navigation. In some embodiments, health care provider GUI 150 may process the database cube output to remove errant data and/or otherwise organize the data. Health care provider GUI 150 may organize the data in a way that is more understandable and accessible to users. For example, a database cube output may include a complete blood count (“CBC”) panel, which often includes dozens of values. Health care provider GUI 150 may display only the CBC values that are elevated, low, or otherwise significant.

Although depicted as separate components in FIG. 1, it should be understood that a component or portion of a component in environment 100 may, in some embodiments, be integrated with or incorporated into one or more other components. For example, all or a portion of machine learning model 130 may be integrated into one or more database cubes 135 or the like. In another example, machine learning model 130 may be integrated into data fabric 125. In some embodiments, operations or aspects of one or more of the components discussed above may be distributed amongst one or more other components. Any suitable arrangement and/or integration of the various systems and devices of environment 100 may be used.

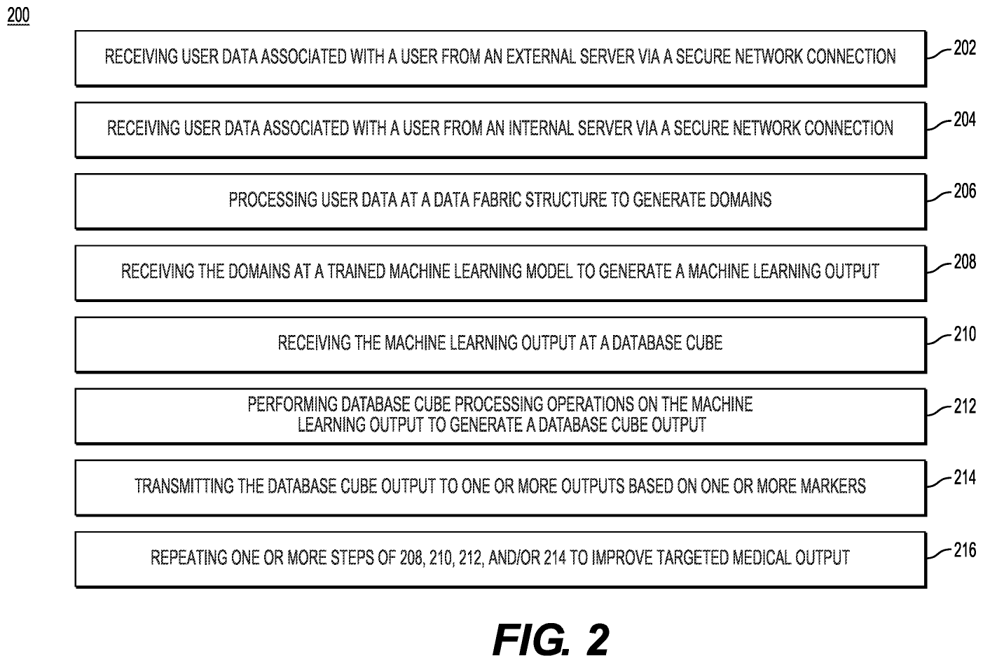

FIG. 2 illustrates an exemplary process for generating secure outputs using machine learning model 130 and database cube(s) 135, according to one or more embodiments. At step 202, data fabric 125 may receive first user data. The first user data may be received via external input 110, discussed above, e.g., data associated with a user from a server external to the data fabric. Data fabric 125 may receive the first user data at a data lake, which may convert the received data into flattened atomic data that may be sorted into domains. The external sever may be associated with one or more health institutions, insurance companies, and/or healthcare entities (e.g., hospital electronic medical records (“EMR”), ambulance logs, urgent care logs, Cigna, Aetna, Anthem, Blue Cross Blue Shield, etc.).

At step 204, data fabric 125 may receive second user data. The second user data may be received via internal input 120, e.g., data associated with a user from an internal server. The internal sever may hold patient-related information, such as information related to a patient's identification information, claims, payments, or the like. Internal input 120 may be stored on a cloud-based data lake associated with data fabric 125, e.g., Microsoft Azure Data Lake, or any suitable storage system.

The first user data and second user data may be transmitted to data fabric 125 via a secure network connection. In some embodiments, a secure network connection may be a connection that is encrypted by one or more security protocols. The secure network connection may ensure the security of data flowing between two or more nodes. A secure network connection may also be a connection that is compliant with the Health Insurance Portability and Accountability Act (“HIPAA”). A secure network connection may satisfy HIPAA requirements if it comprises specific security measures, such as security protections to ensure network confidentiality, integrity, and availability. In some embodiments, the secure network connection may be a secure shell protocol (“SSH”), secure file transfer protocol (“SFTP”), hypertext transfer protocol secure (“HTTPS”), secure hypertext transfer protocol (“S-HTTP”), and so forth. The secure network connection can be any suitable connection. The type of secure network connection for the first user data may be the same or different from the type of connection for the second user data. In any case, the secure network connection used in step 202 may be the same or different secure network connection used in step 204.

At step 206, data fabric 125 processes the user data to generate flattened atomic data that may be sorted into domains. The user data or portions thereof may be transmitted to a staging table, for example a data staging tables database. According to some aspects, the user data is transmitted to the staging table in its raw (e.g. unprocessed) format. Data fabric 125 may process the user data by adding metadata, for example, data obtained from an internal server (e.g., internal input 120), to the data staging tables database. According to some aspects, the metadata may include a time stamp associated with the time the user data was stored on the cloud-based data lake. In some aspects, the data may be claims processing data, for example, data obtained from internal software pertaining to a patient's medical claim records, payment information, or to medical codes associated with a patient's medical care. In some embodiments, the data received or obtained from internal software may be stored on the cloud-based data lake database. According to some aspects, the metadata added to the staging table (e.g. data staging table database) may include a date the data was loaded onto the cloud-based data lake database or the data staging tables database.

Data fabric 125 may modify the user data based on a determined correlation between the user data, the user identification data, and metadata to generate modified user data. Data fabric 125 may extract relevant data from the modified user data. Relevant data may be, for example, data that has previously been used or requested by downstream analytics processes. For example, relevant data may include a user's name, address, contact information, number of hospital or emergency room visits over a time frame, associated medical or claim codes, and other discrete portions of information that may be helpful for data analytics processes. According to some aspects of the disclosure, the relevant data may be extracted from the modified user data using a trained data fabric machine learning model. According to this aspect of the disclosure, the trained data fabric machine learning model may be trained to extract relevant data from the modified user data based on (i) training relevancy data that includes information regarding prior relevant data extracted from prior modified user data associated with other users and (ii) training user data that includes prior relevant data extracted from prior modified user data, to learn relationships between the training relevancy data and the training user data, such that the trained data fabric machine learning model is configured to use the learned relationships to extract modified user data in response to input of the modified user data.

Data fabric 125 may format the relevant data into atomic data, and subsequently store that data on a database, for example, an atomic data database. Atomic data refers to data that typically cannot be broken down into smaller parts. For example, a postal code or zip code, a street address number, a medical claim code, gross revenue, a base salary, unit sales for a product, a username, a password, and so forth. Thus, according to some aspects, all relevant information extracted from the modified user data is separated into atomic data and stored.

Data fabric 125 may generate a plurality of domains based on the atomic data and subsequently store the plurality of domains on a plurality of domain databases. A domain as used herein refers to a collection of values that a data element may contain. For example, a domain may be “lab results,” which may comprise elements including “lab name,” “test name,” “test code,” “test cost,” and “test results.” According to some aspects of the disclosure, one of the plurality of domains may further include one or more sub-domains. For example, “test results” may be a sub-domain, which may further have additional information such as “positive,” “negative,” or “inclusive.” According to aspects of the disclosure, the plurality of domains may include one or more of: a provider domain; a cms domain; a risk domain; a finance domain; a quality domain; a master data domain; a health plan domain; a member domain; a medical claim domain; a claim domain; or a lab result domain. According to some aspects of the disclosure, each domain of the plurality of domains may be stored in the format of one or more of: an .xls file; a csv file; a pipe delimited file, or a text file. By using atomic data to populate the plurality of domains, only information necessary to a particular domain is extracted from the data. Further, a single file format, for example, .xls, may be used across the domains. By formatting the user data into atomic data and sorting that data into domains, downstream product schemas and other software can more easily and quickly pull relevant data for data analytics processes.

At step 208, machine learning model 130 receives and processes, e.g., analyzes, the domains. In some embodiments, machine learning model 130 may also or alternatively process other data (e.g., response data) or any other suitable form of data according to one or more of the techniques or examples discussed herein. Machine learning model 130 may generate machine learning data (e.g., machine learning outputs that may include one or more markers). In some embodiments, machine learning model 130 may be a previously trained model. Training of machine learning model 130 is discussed in further detail below.

According to some aspects, machine learning model 130 may receive domains as an input and may correlate the domains with one or more users and to generate a machine learning output, for each of the one or more users. The machine learning output may include correlated data for each of the one or more users and one or more markers associated with the correlated data for each of the one or more users. Machine learning model 130 may correlate user identification information with untagged healthcare data to map a user to the user's respective data. Machine learning model 130 may receive user data for a plurality of users (e.g., from an external server) and user identification information for the plurality of users (e.g., from an internal server). Machine learning model 130 may match user data to respective user identification information such that each user has her own respective user data associated with her user identifiers.

For example, a user's hospital admittance information may be received from a hospital network and may be tagged with a unique hospital network identifier. The user's identification information may be received form an internal server. Machine learning model 130 may correlate the user's hospital admittance information with the user's identification information based on probabilities of overlap between the hospital admittance information and the identification information. Machine learning model 130 may determine that the biometric data and physical data (e.g., height, weight, etc.) included in the hospital admittance information correlates with the identification information with at least a threshold probability, and may assign the user's hospital admittance information with the same user patient ID that is assigned to the user's identification information. According to some aspects, machine learning model 130 may generate or update a mapping file, mapping user data to the user identification information. According to this implementation, user data may be modified or formatted to generate modified user data that is mapped to the internal user identification and includes metadata indicating a time of upload. According to some aspects, this modified data may be generated using an extract, transform, and load (“ETL”) process on the user data. The ETL process is a data integration process that combines data from multiple data sources into a single, consistent data store that is loaded into a data warehouse or other target system. In this manner, the user data is modified to make extraction of relevant information faster and/or by using less computing resources.

In addition to domains, machine learning model 130 may receive and process, e.g., analyze, response data from the patient. In some embodiments, the patient receives automatic outreach 145 with information about their health, e.g., whether recent blood test results indicate a high likelihood of developing an autoimmune disease. The patient may, in response to receiving outreach, provide response data to machine learning model 130. For example, automated outreach 145 may inform a patient that, based on the patient's latest blood tests, there exists a high probability that the patient will develop a particular autoimmune disease within the next year. The patient may provide information back to machine learning model 130 as to whether an autoimmune disease manifested and, if so, within what time frame it did so. Alternatively or in addition, a sensor associated with a user device (e.g., a patient wearable device having a blood pressure sensor) may automatically provide response data associated with the user to machine learning model 130. Machine learning model 130 can receive this information and use it in an intra-patient (i.e. within a single medical patient, usually over time, rather than between patients) manner to improve the correlations between that patient's data and disease manifestation. Machine learning model 130 could also use the information from the patient to improve correlations between patient data and disease manifestation in an inter-patient manner (i.e. between more than one patients).

In some embodiments, machine learning model 130 may perform one or more machine learning operations on the data. As discussed above, any suitable machine learning technique or combination of techniques, e.g., CNN or RNN, may be used. For example, in some embodiments, machine learning model 130 may use a CNN to analyze data fabric output and generate a treatment recommendation.

In some embodiments, machine learning model 130 may output machine learning data. As discussed above, the machine learning data may include risk assessments, disease diagnoses, treatment options, predictions for a patient's hospital or emergency room admission or readmission, or the like based on correlated user's health information. The machine learning data may include one or more markers generated based on a user's correlated health information, as disclosed herein. In some embodiments, machine learning model 130 may initiate transmission of the machine learning data to any other aspect of environment 100, e.g., to database cube 135. In some embodiments, other aspects of environment 100 may initiate transmission of the machine learning data, e.g., database cube 135 may initiate a pull for transmission of the machine learning data (e.g., machine learning outputs that may include markers) from machine learning model 130.

In some embodiments, the machine learning data may be transmitted for one or more subsequent uses. These uses may include one or more of internal report generation module 140, automated outreach 145, health care provider graphical user interface 150, and/or any other suitable use. In some embodiments, the machine learning data may be stored by any suitable storage system, e.g., an internal storage system or an external system.

At step 210, database cube 135 may receive the machine learning data generated at step 208. In some embodiments, database cube 135 may also receive one or more inputs from any component of environment 100 or from any other suitable source. In some embodiments, database cube 135 may also or alternatively process data from other sources, e.g., data fabric 125, according to one or more of the techniques or examples discussed above.

At step 212, database cube 135 processes the machine learning data and/or any other applicable data received at database cube 135. As discussed above, any suitable database cube processing technique or combination of techniques may be employed. For example, in some embodiments, database cube 135 may use an OLAP to analyze hospitalizations per year for a given patient to determine the likelihood of subsequent hospitalizations. Database cube 135 may generate a database cube output. The database cube output, as discussed above, may be based on relevant fact data, such as machine learning data and/or associated markers. The database cube markers may measure the urgency of the machine learning outputs and determine which parties to transmit the database cube output, based on that urgency. For example, high urgency database cube outputs may be sent to both the medical provider and the patient. In another example, low urgency data may be sent to only the medical provider.

Data cube 135 may process the data received from machine learning model 130 to structure the machine learning data in accordance with a database cube 135 schema. Database cube 135 schema may be determined based on one or more rules associated with database cube outputs. For example, database cube 135 schema may structure the data in a hierarchical system in order of an urgency value associated with a marker or corresponding data. As another example, database cube 135 schema may structure data in a chronological order or in an order based on an intended output recipient (e.g., health care provider, patient, report, etc.).

At step 214, the database cube output is communicated, e.g., to a recipient. Outputs may include one or more of internal report generation module 140, automated outreach 145, health care provider GUI 150, or any other suitable output. One or more markers may be used to identify an output for given database cube outputs.

As discussed above, one or more markers may be used to determine which parties may be informed of a patient's health information, e.g., based on urgency values. In some embodiments, certain markers may signal that different parties may be notified, such as both the patient and the medical provider, just the medical provider, just the patient, or any suitable combination of these parties or others. For example, certain markers, such as a high likelihood of a relapse in relapsing-remitting multiple sclerosis (“RRMS”), may have a high urgency value. Based on the programmed preference settings or dynamic urgency thresholds identified by machine learning model 130, a high-urgency database cube output may be communicated to both the medical provider and the patient. In another example, a marker signaling a high urgency value for an impending relapse of RRMS may trigger cube output to be sent to the medical provider, without being transmitted to the patient. In some cases, medical providers may prefer to review the information before a patient is made aware, and such a preference may be programmed into data cube 135 (e.g., if a marker corresponds to a condition that cannot be improved by immediate patient action, then any outputs based on that marker are first sent to a health care provider). The system may be programmed to the preference of the medical provider, the patient, or any suitable third party.

Data fabric 125 may cause to present, e.g., via a GUI, one or more graphical depictions of data associated with the plurality of domains. For example, the GUI may include a healthcare provider or physician dashboard, and include data pertaining to a total number of members or patients, a percent of members who have completed wellness visits, a number of patients currently in the Emergency Room, and so forth. The number of patients may also be depicted with breakdowns of the type of members, for example, members who are considered senior or over the age of 50+ or new members. Providing this data to a physician in this manner can provide the physicians with tools that allow a doctor to quickly review patient data and see trends or determine who needs to schedule an appointment without the need to conduct file by file reviews or hire staff to conduct such reviews. The GUI may be configured such that icons and/or components are dynamically determined for display, via the GUI, based on available domains for a given patient or patient population. The icons and/or components may be dynamically determined based on data identified to be displayed via the GUI. The data may be identified based on available domain data, based on user request for given data, and/or based on a machine learning output.

At step 216, one or more steps of environment 200 may be repeated. For example, if a patient provides response data, such as in the manner described below, machine learning model 130 may process the response data, generate an updated machine learning output, and transmit that output to database cube 135. Database cube 135 may analyze the updated machine learning output, generate an updated database cube output, and communicate that updated database cube, e.g., to the patient. The steps may be repeated as necessary to improve targeted medical output.

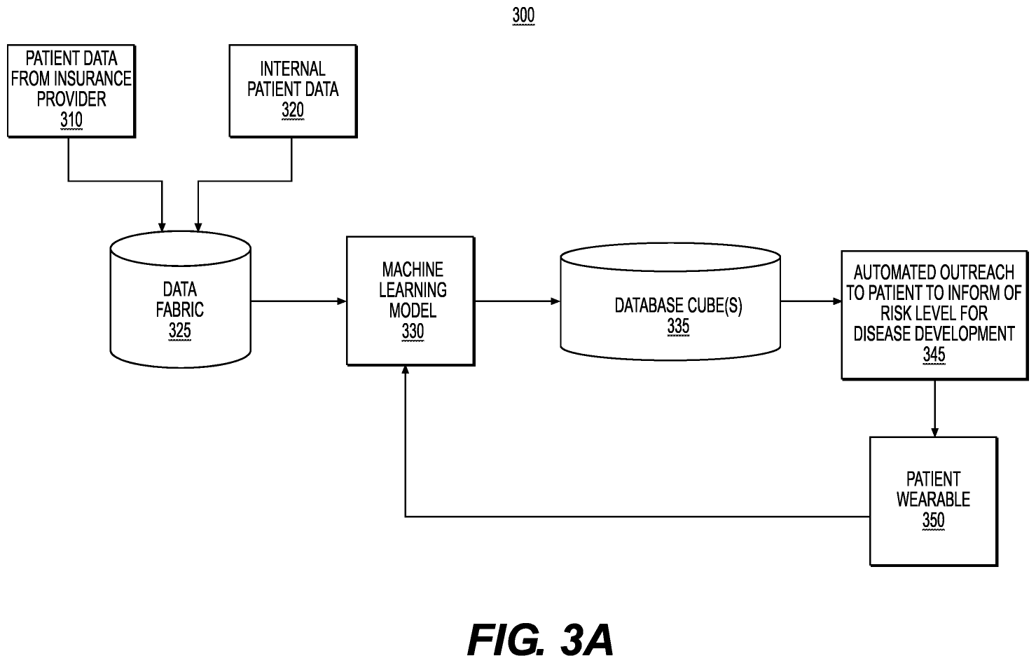

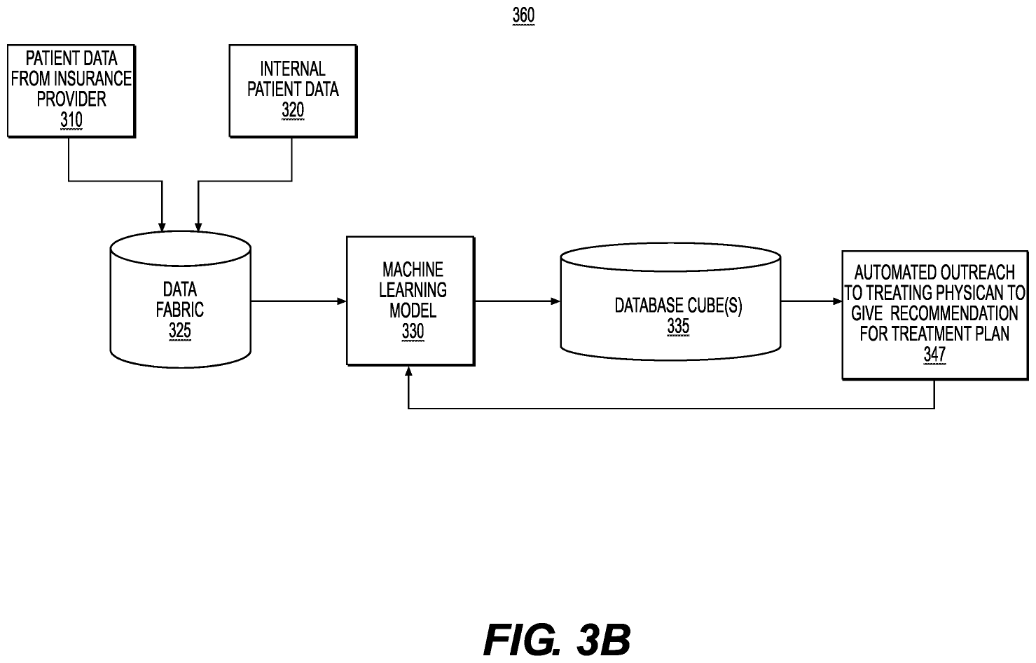

FIG. 3A depicts an exemplary environment 300 for providing automated outputs to a patient. One or more of patient data from insurance provider 310 and/or internal patient data 320, a data fabric 325, a machine learning model 330, a database cube 335, an automated outreach 345 to a patient, and a patient wearable 350 may communicate by any suitable means. Patient data from insurance provider 310 may be similar to external inputs 110 as disclosed herein. Internal patient data 320 may be similar to internal inputs 120 as disclosed herein. For brevity, the structure and process are similar to FIG. 1, but the output of the database cube output is automated outreach 345 to patient. Further, automated outreach 345 to patient may communicate with patient wearable 350 that communicates with machine learning model 330.

The data may be processed as described above by a data fabric 325, machine learning model 330, and a database cube 335. Database cube 335 may determine that certain markers are present in the data and initiate an outreach to one or more outputs. Database cube 335 may communicate with automated outreach 345 to the patient in the manner discussed above. For example, if database cube 335 receives data that shows blood work with particular changes that may indicate the patient is at a high risk of developing Type II Diabetes, database cube 335 may determine that this high-urgency information may be communicated to the patient.

In some embodiments, the patient may receive automated outreach 345 at a user device, such as patient wearable 350. Patient wearable 350 may be any suitable device for receiving information, such as a cell phone or a smart watch. Patient wearable 350 may respond to the input of the database cube output in numerous ways. In some embodiments, patient wearable 350 may allow the patient to input data that may be communicated to machine learning model 330. Alternatively, or in addition, patient wearable 350 may include one or more sensors which may sense health information (e.g., biometric information) of a patient. Patient wearable 350 may transmit that information to be received at machine learning model 330. Machine learning model 330 may generate an updated output if a trigger event occurs, e.g. an increase in heart rate, as determine based on the input data. The trigger event may be determined based on a threshold deviation of a user attribute, e.g., a 15% increase in the patient's heart rate.

In an example, a patient may receive a first automated outreach 345 indicating a moderate risk of a myocardial infarction and a recommendation to make an appointment with the patient's medical provider. The patient may subsequently input data to patient wearable 350, such as increasingly frequent and painful chest pains. The data from patient wearable 350 (“response data”) may be communicated to machine learning model 330. The response data may be processed by machine learning model 330 using methods and techniques discussed above. Machine learning model 330 may generate an updated machine learning output that may include one or more markers indicating a high risk of a myocardial infarction and a recommendation to seek immediate medical care. The updated machine learning output may be communicated to database cube 335. Database cube 335 may analyze the updated machine learning output using methods and techniques discussed above and generate an updated database cube output. The updated database cube output may be based on the one or more markers, such as determining that both the patient and medical provider will receive the second automated outreach. The updated database cube output may be communicated to automated outreach 345 to the patient, patient wearable 350, and/or to the medical provider, depending on the programed database cube marker preferences. This process disclosed herein using environment 300 may be repeated.

In some embodiments, patient wearable 350 may collect data on the patient (e.g., via heart sensors on the patient wearable 350) and provide response data to machine learning model 330. For example, if patient wearable 350 received automated outreach 345 of a patient's high risk of a developing a myocardial infarction, patient wearable 350 may receive a signal via the updated data cube output to collect the patient's heart rate and blood oxygen levels at certain time intervals and communicate that information to machine learning model 330. Depending on the information received, machine learning model 330 may communicate further updated machine learning outputs to database cube 335, which may communicate updated database cube outputs to the respective outputs, e.g., to automated outreach 345 to the patient.

In some embodiments, both the patient and patient wearable 350 may communicate with machine learning model 330. For example, if patient wearable 350 received automated outreach 345 of a patient's high risk of a developing a myocardial infarction, the patient may input data of symptoms experienced while patient wearable 350 collects data on the patient's heart rate and blood oxygen levels. The data from patient wearable 350 may be externally collected (e.g., from a patient) or internally collected (e.g., by the patient wearable 350).

FIG. 3B depicts an exemplary environment 360 for implementing an automated outreach to a medical provider. One or more of the patient data from insurance provider 310 and/or internal patient data 320, data fabric 325, machine learning model 330, database cube 335, and an automated outreach 347 to a medical provider may communicate by any suitable means. Patient data from insurance provider 310 may be similar to external inputs 110 as disclosed herein. Internal patient data 320 may be similar to internal inputs 120 as disclosed herein. For brevity, the structure and process are similar to FIG. 1, but the output of the database cube output is automated outreach 347 to the medical provider.

The data may be processed as described above by data fabric 325, machine learning model 330, and/or database cube 335. Database cube 335 may determine that certain markers are present in the data and initiate an outreach to one or more outputs. Database cube 335 may communicate with automated outreach 347 to the medical provider in the manner discussed above. For example, if database cube 335 receives data and/or markers that indicate blood work with particular changes that may indicate the patient is at a high risk of developing Type II Diabetes, database cube 335 may determine that this high-urgency information may be communicated to the medical provider.

In an example, a medical provider may receive a first automated outreach 347 indicating a patient's moderate risk of developing osteoporosis and a recommendation to discuss this issue with the patient. The medical provider may learn more information about the patient's condition, such as from the patient or another medical provider. The medical provider may subsequently communicate updated data to machine learning model 330, such as the patient breaking a bone as indicated in a medical report. The updated data may be processed by machine learning model 330 using methods and techniques discussed above. Machine learning model 330 may generate an updated machine learning output and one or more new markers that indicate a high risk of osteoporosis and a recommendation to enroll the patient in a weight training-based physical therapy. The updated machine learning output may be communicated to database cube 335. Database cube 335 may analyze the updated machine learning output using methods and techniques discussed above and generate an updated database cube output. The updated database cube output may be triggered based on the one or more new markers, such as determining that both the patient and medical provider will receive the second automated outreach. The updated database cube output may be communicated to automated outreach 347 to the medical provider, and/or to the patient, depending on the programed database cube marker preferences. In some embodiments, the updated database cube output may be communicated to patient wearable 350. This process disclosed herein using environment 360 may be repeated.

The medical provider may communicate the collected data to machine learning model 330 via any suitable means. For example, the medical provider may use a centralized patient portal (e.g., MyChart, FollowMyHealth, or Updox) or a device (e.g., a heart sensor) to input the patient information that may be communicated to machine learning model 330. In some embodiments, the patient data stored by the medical provider may be communicated to machine learning model 330 at certain intervals. In some embodiments, the medical provider may use a centralized patient portal to receive updates from the patient or environment 360, or to communicate information to the patient or machine learning model 330. In some embodiments, the medical provider may configure the centralized patient portal to send information about a given patient to machine learning model 330, for example, every week.

In some embodiments, the systems of FIGS. 3A and 3B may operate concurrently. For example, after the first output to automated outreach 347 to the medical provider, the second output may be to automated outreach 345 to the patient, the patient wearable 350, and/or automated outreach 347 to the medical provider.

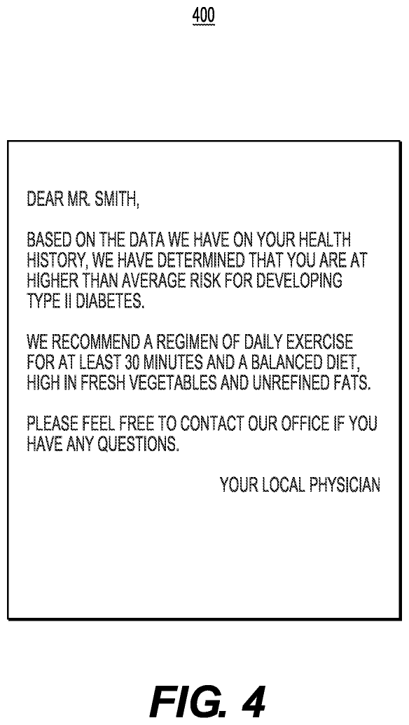

FIG. 4 illustrates an exemplary targeted medical output 400 received by a patient, according to one or more embodiments. In some embodiments, automated outreach 145 may send messages, such as in output 400, to inform patients of their predicted health outcomes, risk for disease development, or any other medical data, as discussed above. The messages may be personalized by medical providers or patients. In some embodiments, patients may configure the system to send all information relevant to a given condition, no information, and/or anything in between. For example, a patient may configure automated outreach 145 to only communicate information as to the risk for disease development and recommended treatment programs, such as output 400 shown in FIG. 4, but not to communicate the likelihood of disease relapse. Patients and medical providers may configure the system to send automated outreach based on the type, frequency, amount, and/or any other relevant factor as determined by the parties' preferences.

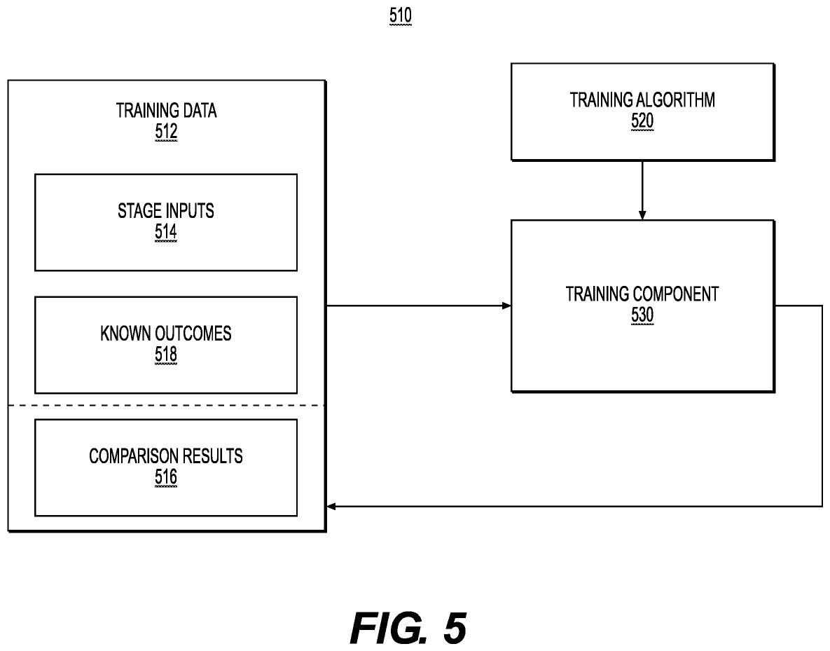

FIG. 5 depicts a flow diagram for training a machine learning model to implement a targeted medical outreach, according to one or more embodiments. One or more of training data 512, stage inputs 514, known outcomes, 518, comparison results 516, a training algorithm 520, and a training component 530 may communicate by any suitable means. One or more implementations disclosed herein may be applied by using a machine learning model. A machine learning model as disclosed herein may be trained using environment 100 of FIG. 1, environment 200 of FIG. 2, environment 300 of FIG. 3A, and/or environment 360 of FIG. 3B. As shown in flow diagram 510 of FIG. 5, training data 512 may include one or more of stage inputs 514 and known outcomes 518 related to a machine learning model to be trained. The stage inputs 514 may be from any applicable source including a component or set shown in FIGS. 1, 2, 3A, and/or 3B. Known outcomes 518 may be included for machine learning models generated based on supervised or semi-supervised training. An unsupervised machine learning model might not be trained using known outcomes 518. Known outcomes 518 may include known or desired outputs for future inputs similar to or in the same category as stage inputs 514 that do not have corresponding known outputs.

Training data 512 and a training algorithm 520 may be provided to a training component 530 that may apply training data 512 to training algorithm 520 to generate trained machine learning model 130. According to an implementation, training component 530 may be provided comparison results 516 that compare a previous output of the corresponding machine learning model to apply the previous result to re-train machine learning model 130. Comparison results 516 may be used by training component 530 to update the corresponding machine learning model. Training algorithm 520 may utilize machine learning networks and/or models including, but not limited to a deep learning network such as Deep Neural Networks (“DNN”), Convolutional Neural Networks (“CNN”), Fully Convolutional Networks (“FCN”) and Recurrent Neural Networks (“RCN”), probabilistic models such as Bayesian Networks and Graphical Models, and/or discriminative models such as Decision Forests and maximum margin methods, or the like. The output of the flow diagram 510 may be a trained machine learning model.

It should be understood that embodiments in this disclosure are exemplary only, and that other embodiments may include various combinations of features from other embodiments, as well as additional or fewer features. For example, while some of the embodiments above pertain to implementing an automated outreach, any suitable activity may be used. In an exemplary embodiment, instead of or in addition to automated outreach to a patient, implementing a targeted medical outreach may include providing input to a medical provider's GUI.

In general, any process or operation discussed in this disclosure that is understood to be computer-implementable, such as the processes illustrated in FIGS. 1, 2, 3A, and 3B, may be performed by one or more processors of a computer system, such any of the systems or devices in the environment 100 of FIG. 1, as described above. A process or process step performed by one or more processors may also be referred to as an operation. The one or more processors may be configured to perform such processes by having access to instructions (e.g., software or computer-readable code) that, when executed by the one or more processors, cause the one or more processors to perform the processes. The instructions may be stored in a memory of the computer system. A processor may be a central processing unit (“CPU”), a graphics processing unit (“GPU”), or any suitable types of processing unit.

A computer system, such as a system or device implementing a process or operation in the examples above, may include one or more computing devices, such as one or more of the systems or devices in FIG. 1. One or more processors of a computer system may be included in a single computing device or distributed among a plurality of computing devices. A memory of the computer system may include the respective memory of each computing device of the plurality of computing devices.

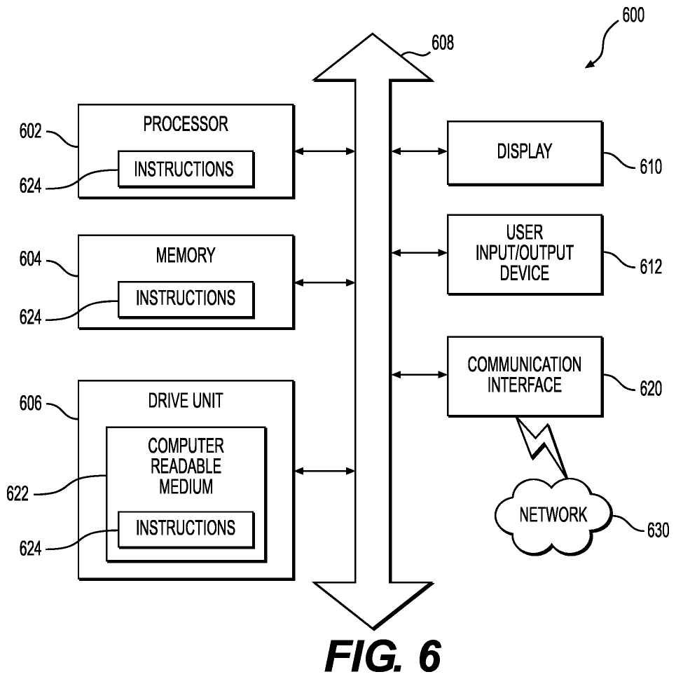

FIG. 6 is a simplified functional block diagram of a computer 600 that may be configured as a device for executing the method of FIG. 2, according to exemplary embodiments of the present disclosure. One or more of a processor 602, a memory 604, a drive unit 606, an internal communication bus 608, a display 610, a under input/output ports 612, a communication interface 620, a computer readable medium 622, instructions 624, and a network 630 may communicate by any suitable means. For example, computer 600 may be configured as data fabric 125, machine learning model 130, database cube 135, and/or another system according to exemplary embodiments of this disclosure. In various embodiments, any of the systems herein may be a computer 600 including, for example, data communication interface 620 for packet data communication. Computer 600 also may include a central processing unit (“CPU”) 602, in the form of one or more processors, for executing program instructions. Computer 600 may include internal communication bus 608, and storage unit 606 (such as Read-Only Memory (“ROM”), Hard Disk Drive (“HDD”), Solid-State Drive (“SSD”), etc.) that may store data on computer readable medium 622, although computer 600 may receive programming and data via network communications. Computer 600 may also have memory 604 (such as Random-Access Memory (“RAM”)) storing instructions 624 for executing techniques presented herein, although instructions 624 may be stored temporarily or permanently within other modules of computer 600 (e.g., processor 602 and/or computer readable medium 622). Computer 600 also may include input and output ports 612 and/or display 610 to connect with input and output devices such as keyboards, mice, touchscreens, monitors, displays, etc. The various system functions may be implemented in a distributed fashion on a number of similar platforms, to distribute the processing load. Alternatively, the systems may be implemented by appropriate programming of one computer hardware platform.

Program aspects of the technology may be thought of as “products” or “articles of manufacture” typically in the form of executable code and/or associated data that is carried on or embodied in a type of machine-readable medium. “Storage” type media include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer of the mobile communication network into the computer platform of a server and/or from a server to the mobile device. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links, or the like, also may be considered as media bearing the software. As used herein, unless restricted to non-transitory, tangible “storage” media, terms such as computer or machine “readable medium” refer to any medium that participates in providing instructions to a processor for execution.

While the disclosed methods, devices, and systems are described with exemplary reference to transmitting data, it should be appreciated that the disclosed embodiments may be applicable to any environment, such as a desktop or laptop computer, an automobile entertainment system, a home entertainment system, etc. Also, the disclosed embodiments may be applicable to any type of Internet protocol.

It should be appreciated that in the above description of exemplary embodiments of the invention, various features of the invention are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment. Thus, the claims following the Detailed Description are hereby expressly incorporated into this Detailed Description, with each claim standing on its own as a separate embodiment of this invention.

Furthermore, while some embodiments described herein include some but not other features included in other embodiments, combinations of features of different embodiments are meant to be within the scope of the invention, and form different embodiments, as would be understood by those skilled in the art. For example, in the following claims, any of the claimed embodiments can be used in any combination.

Thus, while certain embodiments have been described, those skilled in the art will recognize that other and further modifications may be made thereto without departing from the spirit of the invention, and it is intended to claim all such changes and modifications as falling within the scope of the invention. For example, functionality may be added or deleted from the block diagrams and operations may be interchanged among functional blocks. Steps may be added or deleted to methods described within the scope of the present invention.

The above disclosed subject matter is to be considered illustrative, and not restrictive, and the appended claims are intended to cover all such modifications, enhancements, and other implementations, which fall within the true spirit and scope of the present disclosure. Thus, to the maximum extent allowed by law, the scope of the present disclosure is to be determined by the broadest permissible interpretation of the following claims and their equivalents, and shall not be restricted or limited by the foregoing detailed description. While various implementations of the disclosure have been described, it will be apparent to those of ordinary skill in the art that many more implementations are possible within the scope of the disclosure. Accordingly, the disclosure is not to be restricted except in light of the attached claims and their equivalents.

What is claimed is:

1. A method for generating secure targeted outputs using a trained machine learning model and a database cube, the method comprising:
receiving first user data associated with a user from an external server via a first secure network connection;
receiving second user data associated with a user from an internal server via a second secure network connection;
processing the first user data and the second user data at a data fabric structure to generate domains;
receiving the domains as an input at a machine learning model trained to correlate the domains with one or more users and to generate a machine learning output, for each of the one or more users, the machine learning output comprising correlated data for each of the one or more users and one or more markers associated with the correlated data for each of the one or more users;
receiving, at the database cube and for each of the one or more users, the machine learning output;
performing one or more database cube processing operations on the machine learning output to generate a database cube output, the database cube output based at least on the one or more markers associated with the correlated data; and
transmitting the database cube output to one or more outputs, wherein the one or more outputs are identified based on the one or more markers.

2. The method of claim 1, wherein the first user data or the second user data comprises one or more of user institution records, user identification information, or user financial data.

3. The method of claim 1, wherein the first user data or the second user data is in a format of one or more of an .xls file, a csv file, or a text file.

4. The method of claim 1, wherein the external server is associated with a health insurance company, a hospital, or a medical office.

5. The method of claim 1, wherein the machine learning model is trained based on historical user data for a plurality of users and wherein training the machine learning model further comprises:
receiving training data including the historical user data;
receiving outcome data tagged based on the historical user data;
adjusting at least one of weights, biases, or layers of a training model based on the training data and the outcome data; and
outputting the machine learning model based on the trained model.

6. The method of claim 1, wherein the one or more markers are at least one of a risk value, an urgency value, a time frame, a severity prediction, a relapse probability, or a disease indication.

7. The method of claim 1, further comprising:
transmitting the database cube output to a user device, the user device configured to respond to the database cube output to generate response data using one or more sensors;
receiving the response data output from the user device; and
providing the response data to the machine learning model, the machine learning model configured to generate an updated machine learning output based on the response data, wherein the updated machine learning output comprises an updated marker.

8. The method of claim 7, wherein the updated machine learning output is generated based on an occurrence of a trigger event.

9. The method of claim 8, wherein the trigger event is determined based on a threshold deviation of a user attribute.

10. The method of claim 1, wherein an output of the one or more outputs is at least one of an internal report generation module, an automatic outreach, or a health care provider graphical user interface.

11. The method of claim 1, further comprising:
formatting components of a graphical user interface based on the database cube output and the one or more outputs; and
generating the formatted components of the graphical user interface.

12. The method of claim 11, wherein the formatted components of the graphical user interface based on a first database cube output and a first output are different than the formatted components of the graphical user interface based on a second database cube output.

13. A system for generating secure targeted outputs using a trained machine learning model, the system comprising:
at least one memory storing instructions; and
at least one processor executing the instructions to perform a process, the processor configured to:
receive first user data associated with a user from an external server via a first secure network connection;
receive second user data associated with a user from an internal server via a second secure network connection;
process the first user data and the second user data at a data fabric structure to generate domains;
receive the domains as an input at a machine learning model trained to correlate the domains with one or more users and to generate a machine learning output, for each of the one or more users, the machine learning output comprising correlated data for each of the one or more users and one or more markers associated with the correlated data for each of the one or more users;
receive, at a database cube and for each of the one or more users, the machine learning output;
perform one or more database cube processing operations on the machine learning output to generate a database cube output, the database cube output based at least on the one or more markers associated with the correlated data; and
transmit the database cube output to one or more outputs, wherein the one or more outputs are identified based on the one or more markers.

14. The system of claim 13, wherein the external server is associated with a health insurance company, hospital, or a medical office.

15. The system of claim 13, wherein one or more processors are associated with at least one of an internal report generation module, an automatic output coordinator, or a health care provider graphical user interface.

16. The system of claim 13, wherein the processor is further configured to:
format components of a graphical user interface based on the database cube output and the one or more outputs; and
generate the formatted components of the graphical user interface.

17. The system of claim 16, wherein the formatted components of the graphical user interface based on a first cube output and a first output are different than the formatted components of the graphical user interface based on a second cube output.

18. The system of claim 13, wherein the secure network connection is a Health Insurance Portability and Accountability Act (“HIPAA”) compliant connection.

19. The system of claim 13, the system further comprising:
transmit the database cube output to a user device, the user device configured to respond to the database cube output to generate response data using one or more sensors;
receive the response data output from the user device; and
provide the response data to the machine learning model, the machine learning model configured to generate an updated machine learning output based on the response data, wherein the updated machine learning output comprises an updated marker.

20. A method for generating secure targeted outputs based on database inputs, the method comprising:
receiving first user data associated with a user, from a plurality of users, from an external server via a first secure network connection;
receiving second user data associated with the user from an internal server via a second secure network connection;
processing the first user data and the second user data at a data fabric structure to generate domains;
receiving the domains as an input at a database cube for each of the plurality of users including the user;
performing one or more cube processing operations on the domains to generate a database cube output, the database cube output based at least on one or more markers associated with correlated data; and
transmitting the database cube output to one or more outputs, wherein the one or more outputs are identified based on the one or more markers.
